SEQUENCE LISTING (1 sequence)

SEQ ID NO: 1 - protein, 400 aa, Human
EPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

(19) United States
(12) Patent Application Publication
(10) Pub. No.: US 2022/0370554 A1
(43) Pub. Date: Nov. 24, 2022

(54) Calreticulin for Treating or Preventing an Angiogenic Eye Disease

(71) Applicant: UAB BALTYMAS, Vilmius (LT)

(72) Inventors: Rimantas Slibinskas, Vilnius (LT); Evaldas Ciplys, Vilnius (LT)

(73) Assignee: UAB BALTYMAS, Vilnius (LT)

(21) Appl. No.: 17/767,232

(22) PCT Filed: Oct. 7, 2020
(86) PCT No.: PCT/EP2020/078173

§ 371 Date: Apr. 7, 2022

(30) Foreign Application Priority Data
Oct. 8, 2019 (GB) 1914516.8

(51) Int. Cl. A61K 38/17; A61K 9/00; A61P 27/02; A61K 45/06

(52) U.S. Cl. CPC A61K 38/1738; A61K 9/0048; A61P 27/02; A61K 45/06

(57) ABSTRACT

The present invention relates to therapeutic use of calreticulin in treatment of angiogenic eye diseases. Different treatment methods and pharmaceutical compositions comprising calreticulin are disclosed.

### TECHNICAL FIELD

The present disclosure relates to calreticulin protein for use in treating or preventing angiogenic eye disease in a subject and pharmaceutical compositions suitable for said use.

### BACKGROUND

Ocular neovascularization is a major cause of blindness associated with choroidal neovascularization, corneal neovascularization, proliferative diabetic retinopathy, retinal neovascularization, age-related macular degeneration, and neovascular glaucoma. In particular, age-related macular degeneration (AMD) is one of the leading causes of irreversible damage to vision in people over the age of 50 years. This disease alone hits millions of older people, the treatment options are limited, and all require invasive procedures. Moreover, current treatments result in a number of side effects following the uncomfortable, and often painful, intraocular injections. Most of the injectable drugs used are antibodies directed against vascular endothelial growth factor (VEGF). These include Avastin® (bevacizumab), Lucentis® (ranibizumab) and Eylea® (aflibercept). Unfortunately, the positive effects after injection of such a drug fade with time, resulting in the need for further repeated intravitreal injections. Usually, the treatment course includes injections every 4 weeks over a total treatment time of 52 weeks. Still, the final outcome is not very promising as the positive effects of the treatment tend to fade and disappear in the longer term. In particular, it has been reported that in AMD the initial gain in Vision Acuity (VA) was not maintained in the long term (see Wecker et al., 2017). Despite initial gains in vision using anti-VEGF treatment, the mean VA of treated eyes had deteriorated to baseline or worse around the time treatment was discontinued (Gillies et al., 2015).

If it is accepted that anti-VEGF treatment should be continued indefinitely, there is another problem. Injections require large amounts of the antibodies and the cost of one procedure usually exceeds 1000 €. For years, Lucentis®, from Roche and Novartis, and Bayer and Regeneron's Eylea®, have dominated AMD treatment, with combined 2017 sales topping $9 billion. The U.S. government-administered Medicare health plan for seniors spent $3.25 billion in 2016 on Eylea® and Lucentis® alone.

In the light of the above, the need for more effective and less expensive treatment options is indisputable. Moreover, from the patient's point of view, it is desirable that any new treatment options would be non-invasive, painless and provide improved effect in longer term.

However, ocular drug delivery is an extremely challenging area due to the restrictive barrier functionalities of the eye. The eye can be divided into the anterior segment, which is the front third of the eye and includes structures in front of the vitreous, and the posterior segment that includes the vitreous, retina and choroid. Topical administration (TA) is the most desirable route of administration, since it allows for self-administration, is non-invasive, and is the most acceptable to the patient. However, the cornea layers, particularly the epithelium and stroma that are two of the outermost layers of the eye, are considered major barriers for ocular drug delivery (Gaudana et al., 2010). Moreover, the tear film as a buffered aqueous fluid displays a fast restoration time of 2-3 min and most administered eye drops/solutions are washed away within the first 15-30 seconds, resulting in poor bioavailability (<5%) (Barar et al., 2008). As such, topical administration (e.g. in the form of eye drops) is only possible for certain active agents and is not normally effective for the treatment of posterior segment diseases, such as angiogenic eye diseases involving the choroid, retina or macula.

Due to the importance of angiogenesis in tumor growth and metastasis, there has been much research on anti-angiogenic compounds and their potential use in treating cancer. Some of these, such as the antibodies directed against vascular endothelial growth factor, have also been found to work in the treatment of angiogenic eye disease.

One protein and related protein fragment that have been investigated for its anti-angiogenic effects and potential to treat cancer is calreticulin (CRT) and its N-terminal fragment, known as vasostatin, which comprises amino acids 1-180 of calreticulin (VS180). Initial work on vasostatin and calreticulin in the context of tumor growth, reported that vasostatin that had been produced in E. coli as a recombinant protein fused to maltose-binding protein, and calreticulin that had been produced in E. coli as a recombinant protein fused to glutathione S transferase protein, inhibited the proliferation of endothelial cells (fetal bovine heart endothelial cells) in vitro and, inhibited Burkitt tumor growth when injected subcutaneously into mice (Pike et al., 1998, 1999). In a related patent application (WO 00/20577, first published April 2000, and later patent family member US 2005/0208018 A1, published 22 Sep. 2005 (Tosato et al.)) it was reported that natural gel-eluted calreticulin obtained from purified B cell line supernatant also inhibited proliferation of fetal bovine heart endothelial cells. However, the results of further work on the antiangiogenic properties of these compounds has been mixed. Later work by another group was not able to demonstrate any anti-angiogenic effect of CRT in a co-culture angiogenesis assay using native CRT isolated from human placenta (Friis et al., 2003). Further, several publications over the past decade have reported that CRT was found to promote tumor progression, as increased level of CRT contributed to cancer metastasis in gastric, pancreatic, prostate, and ovarian cancers, to name just a few (e.g. Sheng et al., 2014; for many summarized sources see review of Eggleton et al., 2016). Further, Chen et al., (2009) reported that CRT overexpression enhanced angiogenesis, facilitating proliferation and migration of gastric cancer cells.

Studies on angiogenesis in the context of the eye reported that vasostatin suppressed the progression of induced corneal neovascularization and induced choroidal neovascularization lesions in rat models after topical application in the form of eye drops (Wu et al, 2005; Sheu et al., 2009). However, it was reported that there were a number of problems encountered with recombinantly expressed VS180, mostly attributed to high molecular weight of this construct, namely: (i) VS180 was poor in delivering to cells; (ii) VS180 is not easy to bind with vascular endothelial cells, and thus, the efficiency of inhibition on angiogenesis was low; (iii) the solubility and stability of the VS180 was low in water, so that sediments usually occurred when the VS180 was dissolved in water as being manufacturing into eye drops (Tai et al., 2013). Therefore, the recombinantly expressed VS180 required a tagged protein, such as thioredoxin (TRX), to increase the solubility. However, a thioredoxin-combined recombinantly expressed VS180 (TRX-VS180) induced immune responses in hosts, including red eyes and itch, when it was delivered to individuals, and therefore still performed poorly in practical use (Tai et al., 2013).

It was further tried to reduce the expressed VS fragment to shorter, but still effective peptides. It was found that a peptide fragment of 48 residues, vasostatin 48 (VS48), which consists of residues 133-180 of CRT, is functional in the form of eye drops when expressed as a fusion protein TRX-VS48 (Bee et al., 2010). It was used to treat laser-induced choroidal neovascularization (CNV) in rat eyes. Again, TRX-VS48 was shown to induce negative immune response in rabbits and it was offered to use VS48 peptide alone, without a fusion partner (Tai et al., 2013). The same group recently was able to reduce a size of still active CRT anti-angiogenic domain (CAD) fragment to a peptide of 27 residues, comprising residues 137-163 of CRT, referred to as CAD-like peptide 27 (CAD27). It was produced as a cyclic peptide by chemical synthesis and was still active in the form of eye drops in a rat model of laser-induced CNV, although at much higher concentrations than was previously shown for CRT fragments VS180 and VS48 (Bee et al., 2018). The authors report that CAD27 is advantageous over VS180 and VS48 as it possesses a low molecular weight that allows it to have better retinal or transscleral penetration to the posterior segment when administered through intravitreal injection and topical administration, respectively.

### SUMMARY

Calreticulin for use in treatment or prevention of angiogenic eye disease is described herein. In particular, the present invention provides calreticulin for use in treating or preventing angiogenic eye disease in a subject, wherein the calreticulin is comprised in a pharmaceutical composition.

In a second aspect the present invention provides a method of treating or preventing angiogenic eye disease in a subject, comprising administering an effective amount of the calreticulin to the eye of the subject, wherein the calreticulin is comprised in a pharmaceutical composition.

The invention also provides experimental data which demonstrates that calreticulin (CRT) is effective in the treatment of angiogenic eye disease. In particular, the data provided shows that calreticulin is effective in treating choroidal neovascularization, when administered by intravitreal and topical administration. These results are highly unexpected as to the best of the inventors' knowledge there is no data on CRT crossing the barriers separating different organs or, particularly, some body surface epithelium barrier, and there is a widespread opinion among the experts in the field that CRT is not able to cross any epithelium barrier. Moreover, as noted above, Tai et al. (2013) have shown that vasostatin worked poorly due to its high molecular weight resulting in multiple problems. Therefore, their group have put significant efforts to reduce size of the fragment to as small a peptide as possible. There is no hint or any indication in their work that a bigger protein fragment or protein could be effective.

Contrary to this, the inventors have surprisingly shown that the full-length high molecular weight calreticulin protein is able to cross the different layers of the eye and be effective after both intravitreal and topical administration. Most surprisingly, the topical administration of CRT onto the eye showed more pronounced positive effect than that of vasostatin fragment reported by the group of Tai (over 70% reduction of CNV lesions in comparison to just about 50% reduction in the case of vasostatin in the previous reports). Due to the relative ease of topical administration (compared to intravitreal injection) these findings are of great importance.

In a third aspect the present disclosure provides a pharmaceutical composition comprising calreticulin and one or more of buffering agents, wherein the pharmaceutical composition is formulated for ocular administration.

In a fourth aspect the present invention provides pre-filled intravitreal syringe comprising the pharmaceutical composition described above.

In a fifth aspect the present invention provides an eye drop bottle comprising the pharmaceutical composition described above and a nozzle for dispensing a metered dose of the pharmaceutical composition to the eye.

In a sixth aspect the present invention provides a method of producing a pharmaceutical composition formulated for ocular administration as described above in relation to the third aspect comprising the steps of:
(a) transforming a yeast cell with a nucleotide sequence comprising a coding sequence encoding a polypeptide comprising a native calreticulin signal sequence and a calreticulin;
(b) culturing the yeast cell under conditions such that the calreticulin is expressed in secreted form;
(c) extracting the calreticulin from the culture medium;
(d) using the calreticulin to make the pharmaceutical composition formulated for ocular administration.

Preferred features of the invention are defined in the dependent claims.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Nor is the claimed subject matter limited to implementations that solve any or all of the disadvantages noted herein.

### BRIEF DESCRIPTION OF THE DRAWINGS

To assist understanding of the present disclosure and to show how embodiments may be put into effect, reference is made by way of example to the accompanying drawings in which:

FIG. 1 is a graph showing the percentage of CNV lesions at each follow-up time point. The results for intravitreal (IVT) injections of Eylea® and CRT, and the results for IVT and TA of PBS vehicle, are shown by dashed lines. The results for TA of CRT at different concentrations are shown by solid lines. Open symbols indicate vehicle controls (PBS), whereas closed (black) symbols show treatment with included compounds (Eylea® and CRT).

FIG. 2 is a graph showing the percentage of cured CNV lesions, showing treatment effects at each follow-up time point.

FIG. 3 is a graph showing mice body weight for the different treatment groups. Data are presented as mean±SD of 4 mice per group). Data was statistically analysed by Two-Way ANOVA followed by Sidak's posthoc test. No significant difference was observed between treatment groups.

FIG. 4 is chart showing the differences between different treatment groups in vascular leakage. Results are provided as percentage changes in vascular leakage areas at the end of treatment (Day 14) compared to baseline (Day 0). Columns represent mean changes, whereas error bars show SEM.

FIG. 5 provides twelve representative images obtained from imaging session of a single eye (mouse no. 22, Vehicle-treated (IVT) group) on day 5. The first image in the series (top row, far left) is an infrared reflectance image focused at the level of the superficial retina layer and taken prior to the fluorescein injection. Images 2 to 6 (top row, second from left to top row, far right) are fluorescein angiography (FA) images focused at the retinal level and acquired immediately after s.c. administration of fluorescein at an interval of 60 sec. The seventh image in the series (bottom row, far left) is an infrared reflectance image focused at the level of the choroid and taken prior the fluorescein injection. Images 8-12 (bottom row, second from left to bottom row, far right) are FA images focused at the choroid level and taken immediately after fluorescein injection at interval of 60 sec. Fluorescein leakage at the laser spots increases throughout the imaging session (5 min).

FIG. 6 provides a representative volume intensity projection (VIP, fundus of the eye) and associated B-scan images acquired from each lesion at different follow-up time points (mouse no. 16, Eylea®-treated group). Each row of B-scan images show development of CNV at each lasered spot (outlined in circles) at different time points of imaging, where the top row of B-scan images relate to upper circle in the left hand image of the Figure, the middle row of B-scan images relate to middle circle in the left hand image of the Figure, and the lower row of B-scan images relate to the lower circle in the left hand image of the Figure.

FIG. 7 provides representative images of the FA analysis with examples of mice from different treatment groups. FA images were taken after 5 min. of fluorescein injection with focus both at the level of the superficial retina and at the choroid level of the same eye of the individual mice from each treatment group at indicated time points of the study.

FIG. 8 provides an SDS-PAGE gel photograph showing CRT stability at different pH after 3 months incubation at +22° C. Lanes with samples are indicated as follows: M—protein molecular weight marker; 1—control CRT protein sample taken before incubation (0 h); 2—CRT after incubation in succinate buffer, pH 5.0; 3—CRT in succinate buffer, pH 6.0; 4—CRT in 100 mM phosphate buffer, pH 7.0; 5—CRT in Tris buffer, pH 8.0; 6—CRT in Tris buffer, pH 9.0; 7—CRT in Tris buffer with 150 mM NaCl, pH 7.5; 8—CRT in 100 mM phosphate buffer with 150 mM NaCl, pH 7.4; 9—CRT in 100 mM phosphate buffer with 150 mM NaCl, pH 7.4 (heat shock for 3 min. at 85° C. was done before incubation at RT).

FIG. 9 provides an SDS-PAGE gel photograph showing CRT stability at different pH after 15 days incubation at +37° C. Indication of samples in gel lanes is the same as in FIG. 8.

FIG. 10 provides an SDS-PAGE gel photograph showing CRT stability at different pH after 9 or 10 days incubation at +37° C. Indication of samples in gel lanes M and 1-8 is the same as in FIG. 8 (samples in lanes 2-8 were taken after 9 days incubation at +37° C.). On the lane 9, CRT sample in the 10 mM phosphate buffer with 150 mM NaCl, pH 7.4 was loaded after 10 days incubation at +37° C. (without pre-heating at higher temperature before the incubation).

FIGS. 11A to 11C provide photographs of SDS-PAGE gels showing CRT stability at different protein concentrations in PBS buffer (pH 7.4) after 9 months of storage at different temperatures. FIG. 11A, FIG. 11B and FIG. 11C show gels with CRT protein taken from solutions with its concentrations of 2.5 μg/ml, 25 μg/ml and 250 μg/ml, respectively. Samples in lanes are indicated as follows: M—protein molecular weight marker; St.—control CRT sample taken from solution with the same protein concentration at the starting point of stability study (0 days); Bl.—blank lane; on other lanes the temperatures are indicated at which CRT samples were incubated for 9 months.

FIGS. 12A to 12C are graphs showing the percentage of intact CRT form in different temperatures at different protein concentrations, showing protein integrity and stability in solution at each follow-up time point. The percentages were determined by densitometrical scanning of SDS-PAGE gels. FIG. 12A, FIG. 12B and FIG. 12C show CRT stability at its different concentrations of 2.5 μg/ml, 25 μg/ml and 250 μg/ml, respectively. For each concentration, stability study was performed in solution at three different temperatures of +5° C., +22° C. and +37° C. for 9 months (270 days) as indicated in graphs.

### DETAILED DESCRIPTION

As mentioned above, the present disclosure relates to the medical use of calreticulin in treating or preventing angiogenic eye disease in a subject. Similarly, the present disclosure provides a method of treating or preventing angiogenic eye disease in a subject, comprising administering to the subject an effective amount of calreticulin.

An angiogenic eye disease is a disease that involves abnormal angiogenesis in the eye, causing an impairment of tissue function (e.g. a loss of vision). These diseases can involve one or more of choroidal neovascularisation, corneal neovascularization and retinal neovascularization. Preferably the angiogenic eye disease is one which comprises choroidal neovascularization.

The angiogenic eye disease may be selected from macular degeneration, proliferative retinopathy, proliferative diabetic retinopathy, neovascular glaucoma, retrolental fibroplasia, and corneal neovascularization (e.g. corneal neovascularization secondary to infectious or inflammatory processes). Preferably the angiogenic eye disease is selected from proliferative diabetic retinopathy, macular degeneration and neovascular glaucoma. Most preferably the angiogenic eye disease is wet age-related macular degeneration (wet AMD).

The subject can be any mammal, including a human, non-human primate, or a domesticated mammal such as a cat or a dog. Preferably the subject is a human.

Calreticulin is an endoplasmic reticulum protein that is found in a wide range of species and has a highly conserved sequence. In particular, in humans the calreticulin protein is a 400 amino acid protein having a molecular weight of 46 kDa.

The calreticulin may be described as a mature protein (and not the protein precursor), i.e. one which has undergone post-translational modification and in particular has had the translocation signal removed. In humans the translocation signal is a 17 amino acid hydrophobic N-terminal signal sequence which is cleaved off the 417 amino acid protein precursor. The sequence of human calreticulin precursor (i.e. which includes the 17 amino acid translocation signal) can be found in the UniProt Database at UniProtKB—P27797. The mature human calreticulin protein has SEQ ID NO: 1, which is the sequence of UniProtKB-P27797 minus the 17 amino acid translocation sequence.

The calreticulin may be described as a full-length protein, meaning it is not simply a fragment of calreticulin like vasostatin, but retains the original length of the wild-type mature calreticulin protein, e.g. it consists of the amino acid sequence of SEQ ID No: 1. Alternatively, the calreticulin of the invention may include the removal or addition of one to fifty amino acids, preferably no more than one to twenty amino acids and most preferably no more than one to ten amino acids, at the N- or C-terminals of the protein, provided that the calreticulin with the addition/deletion retains the function of the protein described herein, e.g. in a side-by-side comparison in an vitro assay suitable to determine the ability to treat angiogenic eye disease the calreticulin with the addition/deletion has an effect to at least the same degree (+/−10%) as the corresponding wild-type mature calreticulin (such as a calreticulin having SEQ ID NO: 1). Suitable in vitro assays are cell proliferation assays (using cell lines such as, for example, human umbilical vein endothelial cells (HUVEC), human microvascular endothelial cells (HUMVEC) or normal human dermal fibroblasts (NHDF)), co-culture assays (using combinations of cells lines such as HUVEC and NHDF, or HUMVEC and NHDF), and (trans)migration or chemotactic assays. Preferable the in vitro assay is a cell proliferation assay. Preferably the removal or addition of amino acids is an addition, such as an addition of a protein tag. More preferably the removal or addition of amino acids is an addition of a protein tag at the N-terminus.

In some examples of the present invention the calreticulin may comprise or consist of the amino acid sequence of SEQ ID NO: 1 or an amino acid sequence that is at least 85% identical or at least 90% to SEQ ID NO: 1. Preferably the calreticulin comprises or consists of the amino acid sequence of SEQ ID NO: 1 or an amino acid sequence that is at least 95% identical to SEQ ID NO: 1. More preferably the calreticulin comprises or consists of the amino acid sequence of SEQ ID NO:1 or an amino acid sequence that is at least 98% or at least 99% identical to SEQ ID NO: 1. In particular, variants of calreticulin are known from homologous sequences from different animals and from non-disease causing polymorphisms already known in the art. Suitable variants can also be made based on single amino acid substitutions, particularly conservative substitutions, that retain the function described herein (for example as determined by the in vitro assay(s) indicated above).

The calreticulin may be obtained from eukaryotic cells, and in particular may be obtained by recombinant expression in eukaryotic cells. Preferably the calreticulin is prepared by recombinant expression in yeast cells, such as Saccharomyces cerevisiae or Pichia pastoris. In particular, the calreticulin may be prepared using recombinant protein production technology based on the secretion of native recombinant protein to the culture medium after expression of human calreticulin precursor including its native signal sequence as described in Čiplys et al., 2014 and 2015, both of which are incorporated herein by reference in their entirety.

In certain examples at least 75%, at least 80%, at least 85%, at least 90%, at least 95% of the calreticulin in the pharmaceutical composition is in monomeric form. Preferably at least 90%, more preferably 95%. Monomeric form of the calreticulin in the pharmaceutical composition can be checked by non-denaturing PAGE, such as native PAGE or blue native PAGE as demonstrated in Čiplys et al., 2015, and by SE-HPLC (Size-exclusion high performance liquid chromatography).

Specifically, the present inventors consider that the yeast secreted recombinant CRT is structurally different from the prior art recombinant CRT used in prior art studies such as those described for example by Pike et al., (1999) which is produced as an intracellular protein in the bacterial E. coli. Such proteins do not have the hydrophobic N-terminal signal sequence removed and there is a risk that expression of a complex eukaryotic protein, such as calreticulin, in the cytosol of prokaryotic cells as a fusion to a bacterial protein tag such as MBP or GST may affect the structure of the recombinant product. Moreover, the present inventors have found that in their hands preparation of recombinant CRT in E. coli resulted in a product with a high degree of oligomerized and dimerized protein. In contrast to recombinant CRT produced in E. coli, the present inventors have found that human CRT secreted and purified from yeast culture medium is exclusively monomeric protein (Čiplys et al., 2015).

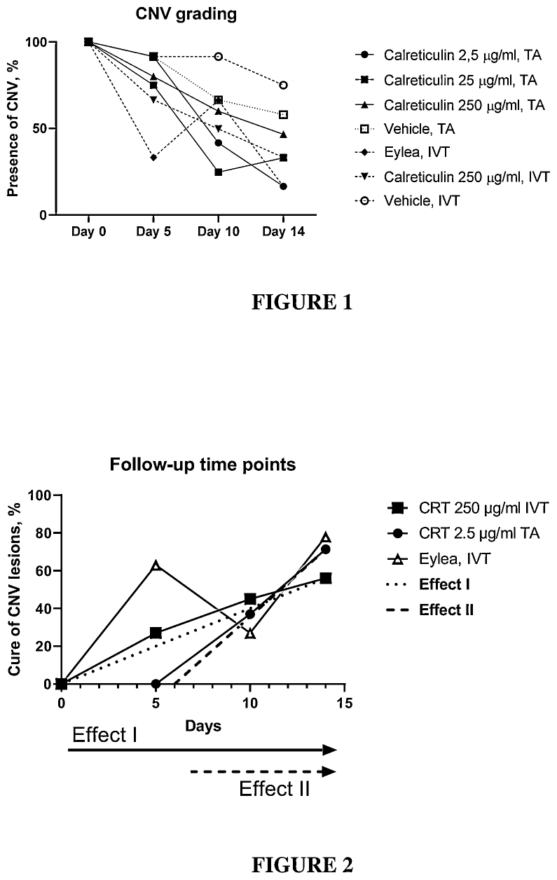

Without wishing to be bound by theory, the present inventors consider that treatment and prevention of angiogenic eye diseases demonstrated herein may be related to a direct inhibition of endothelial cell proliferation and differentiation or an indirect inhibition of angiogenesis or a correction of abnormal angiogenesis and its pathological consequences in vivo, perhaps via multiple pathways induced by the administration of the calreticulin. In the present work described in Example 1, it is shown that the CRT showed strong positive effects in a mouse model of choroidal neovascularization after intravitreal (IVT) injection and after topical application (TA) in the form of eye drops, comparable to that of intravitreal injection of the drug Eylea®, which was used as a positive control (see results shown in FIG. 1).

It is noteworthy, that CRT was used at a dose of just 500 ng/eye (2 μL per eye intravitreal injection), whereas the dose of Eylea® was 40 μg/eye (2 μL per eye intravitreal injection), i.e. CRT was used at 80 times lower doses than the positive control drug. The inventors expect that comparison of IVT injections of CRT vs. Eylea® at equal concentrations or molar ratio will show that CRT is several times more efficient. For the TA, all topically administered CRT groups showed better effect compared to vehicle group. Lower concentrations of topically administrated CRT were more effective than higher concentration, with the most positive effect observed at the highest 100× dilution (working concentration 2.5 μg/ml; dose 12.5 ng/eye, 5 μL per eye topical administration, repeated three times daily).

Without wishing to be bound by theory, the inventors consider that the higher effectiveness of TA of CRT at lower doses may be explained by intrinsic properties of the protein. It has been shown that CRT begins to dimerize and oligomerize at physiological temperatures of 37-40° C. (Jørgensen et al., 2003; Mancino et al., 2002). Moreover, oligomers of CRT are not reversible to monomers at the native physiological conditions (Jørgensen et al., 2003). At the higher concentration the CRT may dimerize/oligomerize faster, due to increased availability of the protein molecules for interaction between each other, and such dimers/oligomers may be too large to enter into the eye after TA. Therefore, lower working concentrations which maintain the protein predominantly in the monomeric form may be necessary in order to achieve the best therapeutic effect in the case of topical administration.

To better visualize and compare observed effects, the main data is redrawn in FIG. 2, which shows cured CNV lesions (in % from total lesions induced by laser) normalized to the effect of administration of the vehicle alone (IVT compounds to IVT vehicle, and TA CRT application to the TA vehicle, respectively). It can be noticed that positive effect of Eylea® fades after 5 days, and restores later, after 10 days. In the case of CRT applications, positive effects are more stable and continuous, irrespective of the way of protein administration (FIGS. 1 and 2).

Accordingly, the use described herein may comprise administration of the pharmaceutical composition comprising the CRT by intravitreal injection or topically. Preferably the use comprises topical administration (TA). In some examples, where the pharmaceutical composition is to be administered by intravitreal injection into the eye the composition may comprise calreticulin at a concentration of 25 μg to 50 mg/ml. In some examples, where the pharmaceutical composition is to be administered topically to the eye, e.g. in the form of eye drops, the pharmaceutical composition may comprise calreticulin at a concentration of 25 ng/ml to 250 μg/ml, preferably 25 ng/ml to 200 μg/ml.

Suitable volumes for administration by topical administration or intravitreal injection will depend on the size of the eye to be treated, and are known in the art. For human eyes, intravitreal injections can be in the range of 0.01 ml to 0.1 ml, but are typically in the range 0.04 to 0.06 ml. For topical administration to human eyes, volumes of 5 to 70 μl can be used.

The work described in Example 1 further revealed different modes of action with important implications for the treatment of angiogenic eye diseases. The therapeutic activity of the CRT may be separated into the two parallel effects: “Effect I” that follows application of high CRT concentrations (mainly after IVT injection) and starts immediately; and “Effect II”, which is induced by small CRT concentrations and starts about a week later, after beginning of treatment by TA (FIG. 2).

It is further noted that Effect I following IVT administration of CRT and the Effect II induced by TA of CRT are not just different in time, but also have different pace. IVT injection results in immediate treatment effect but occurs at slower rate. Whereas, TA of CRT reveals significantly delayed therapy, however this effect develops faster when it starts (FIG. 2). At the end of treatment courses (at the day 14) both IVT (single injection) and TA (multiple doses of drops) administrations result in similar amount of the CRT protein used (about 500 ng per mouse eye), and the Effect II finally shows more CNV lesions totally cured (>70%) than the Effect I (>50%). Therefore, non-invasive and painless TA of CRT eye drops reveals to be serious treatment option for neovascularizations related to various angiogenic eye diseases. Further, in addition to the finding of the therapeutic use of CRT protein for treatment of angiogenic eye diseases, the results suggest that Effects I and II are different and their positive impacts can be additive and can occur side-by-side.

As a result, according to the present disclosure the medical use described herein may include a combination of topical administration and intravitreal injection. For example, the pharmaceutical composition comprising calreticulin may be administered topically to a subject who has received at least one intravitreal injection of a pharmaceutical composition comprising calreticulin. Alternatively, the pharmaceutical composition comprising calreticulin may be administered by intravitreal injection to a subject who has received a topical administration of a pharmaceutical composition comprising calreticulin.

Moreover, the method of treatment or prevention of angiogenic eye disease in a subject described herein may comprise administering to the subject an effective amount of calreticulin by intravitreal injection and administering to the subject an effective amount of calreticulin by topical administration.

In some examples, the medical use of the calreticulin comprises combined, separate or sequential use with an anti-angiogenic agent, preferably where the calreticulin is comprised in a pharmaceutical composition that is administered by topical administration. Similarly, the method of treatment or prevention may comprise administering in combination, sequentially or separately, an anti-angiogenic agent. In particular, the anti-angiogenic agent is preferably an inhibitor of vascular endothelial growth factor (VEGF), more preferably selected from bevacizumab, ranibizumab, and aflibercept. Most preferably the anti-angiogenic agent is aflibercept.

Specifically, it is noted that the experimental results show that the approved anti-VEGF drug Eylea® exhibited an effect that is distinct from both CRT applications during the study's time points suggesting different mode of action. Accordingly, it is expected that the combination of other anti-angiogenic agents, and specifically inhibitors of vascular endothelial growth factor such as Eylea® (aflibercept) will have a synergistic effect, particularly where the combination is with TA of CRT in the form of eye drops since in this case CRT-specific healing of CNV lesions starts at the time when Eylea®'s effect begins to fade.

As noted above the present disclosure also provides pharmaceutical compositions comprising the calreticulin described above which is suitable for use in the eye, and in particular which is formulated for ocular administration e.g. formulated for intravitreal injection or formulated for topical administration to the eye, such as eye drops. Preferably the pharmaceutical composition is formulated as eye drops.

In some examples the pH of the pharmaceutical composition is in the range of 7.0 to 9.0, preferably 7.0 to 8.2, more preferably 7.2 to 8.0. Preferably, where the pharmaceutical composition is formulated for topical administration to the eye the pH of the pharmaceutical composition is 7.2 to 7.6.

In some examples the one or more buffering agent is a phosphate salt with a concentration for buffering eye drops in the range of 5 mM to 20 mM (preferably less than 20 mM) and the pH of the pharmaceutical composition is in the range of 7.0 to 7.6, or preferably 7.2 to 7.6. In an alternative example the one or more buffering agents is Tris at a concentration range of 10 mM to 130 mM and the pH of the pharmaceutical composition is in the range of 7.2 to 8.5, preferably 7.2 to 8.2.

The pharmaceutical composition may have a concentration of calreticulin as described herein in relation to the medical use of the first and second aspects of the invention. The calreticulin of the pharmaceutical composition may be as described herein in relation to the medical use in the first and second aspects of the invention In some examples, the pharmaceutical composition may consist of the calreticulin and a PBS or Tris buffer. In alternative examples, the pharmaceutical composition may comprise one or more additional components selected from the group consisting of a surfactant, a tonicity adjusting agent, a preservative or a penetration enhancer. In particular, the pharmaceutical composition may comprise saline at physiological concentrations.

In some examples the pharmaceutical composition may be frozen or lyophilized.

According to the fourth and fifth aspects of the disclosure the pharmaceutical composition may be packaged for storage and use. Specifically, the present disclosure provides a pre-filled syringe for intravitreal injection comprising the pharmaceutical composition described above. Further the present disclosure provides an eye drop bottle or dispenser comprising a detachable cap, the pharmaceutical composition described above, and a nozzle for dispensing a metered dose of the pharmaceutical composition to the eye. In particular, the volume of the metered dose may be in range from 5 to 70 μl, preferably in the range of 5 to 55 μl. The eye drop bottle or dispenser may have a volume of 10 μl to 10 ml, preferably of 20 μl to 10 ml. The bottle or dispenser may contain a volume sufficient to be able to administer one metered dose of from 5 to 70 μl to each eye. Alternatively, the bottle or dispenser may contain a volume sufficient to be able to administer a plurality of metered doses to each eye. In this example, the cap of the bottle or dispenser can be reattached over the nozzle so that the eye drop bottle or dispenser can be re-used over a time period of a number of days or weeks.

In a sixth aspect the present invention provides a method of producing a pharmaceutical composition formulated for ocular administration as described above in relation to the third aspect comprising the steps of:
(a) transforming a yeast cell with a nucleotide sequence comprising a coding sequence encoding a polypeptide comprising a native calreticulin signal sequence and a calreticulin;
(b) culturing the yeast cell under conditions such that the calreticulin is expressed in secreted form;
(c) extracting the calreticulin from the culture medium;
(d) using the calreticulin to make the pharmaceutical composition formulated for ocular administration.

In particular, as mentioned elsewhere herein, the calreticulin of the present invention may be prepared according to the methods described in Čiplys et al., 2014, 2015, which are expressly incorporated by reference herein in their entirety, as indicated above. Further, the method of the disclosure utilizes the calreticulin in the manufacture of the pharmaceutical composition formulated for ocular administration according to the third aspect of the disclosure and described in more detail above.

The following are intended as examples only and do not limit the present disclosure.

### EXAMPLES

### Example 1

This study was performed to compare the anti-angiogenic effects of aflibercept (Eylea®) and calreticulin on choroidal neovascularization in the mouse laser-induced choroidal neovascularization (CNV) model.

Three laser burns were used to induce CNV in male C57BL/6JRj mice (sourced from commercial vendor, Janvier Labs, France). Study compounds were administered either intravitreally once immediately after CNV induction or topically, three times per day, starting from day 0. Aflibercept (Eylea®) was used as a reference compound and administered intravitreally at a dose of 40 μg per eye. Mice were followed for 14 days using in vivo imaging. In vivo imaging was performed at the baseline, immediately after induction of CNV on day 0, on the follow-up day 5, on the follow-up day 10 and on the follow-up day 14.

The growth of subretinal blood vessels was recruited from the choroid by perforating Bruch's membrane using diode laser. Immediately after the lasering unilateral intravitreal (IVT) administrations of treatment compounds was performed for mice in IVT treatment groups. The contralateral eye remains untouched in all mice and served as control. The mice were followed using in vivo imaging (fluorescein angiography (FA) and spectral domain optical coherence tomography (SD-OCT)) for 14 days after the initial laser application.

A total of 29 mice were used in the study. The following treatment arms were used in the study:
Group 1: CNV+PBS, 2 μL per eye intravitreal injection (n=4)
Group 2: CNV+Eylea®, dose 40 μg/eye, 2 μL per eye intravitreal injection of a 20 mg/ml solution (n=4)
Group 3: CNV+Calreticulin, dose 500 ng/eye, 2 μL per eye intravitreal injection of a 250 g/ml solution (n=4)
Group 4: CNV+Calreticulin, dose 12.5 ng/eye, 5 μL per eye topical administration of a 2.5 g/ml solution (n=4)
Group 5: CNV+Calreticulin, dose 125 ng/eye, 5 μL per eye topical administration of a 25 g/ml solution (n=4)
Group 6: CNV+Calreticulin, dose 1250 ng/eye, 5 μL per eye topical administration of a 250 g/ml solution (n=5)
Group 7: CNV+PBS, 5 μL per eye topical administration (n=4)
   All animals were treated in accordance with the ARVO Statement for the Use of Animals in Ophthalmic and Vision Research and the EC Directive 86/609/EEC for animal experiments.

Male C57BL/6JRj mice aged 8 weeks old, were purchased from Janvier Labs (France). Animals were housed at constant temperature (22±1° C.) (see detailed environmental conditions in Table 1) and in a light-controlled environment (lights on from 7 am to 7 pm) with ad libitum access to food and water. Experiments commenced after a minimum of 1 week quarantine and acclimatization in the vivarium. To monitor body weight the mice were weighed at baseline, before the CNV induction on day 0 and during each in vivo imaging time point.

CNV Induction

All mice were anesthetized by intraperitoneal injection of ketamine (37.5 mg/kg; Ketaminol Vet, Intervet Oy MSD Animal Health, Espoo, Finland) and medetomidine (0.45 mg/kg; Domitor, Orion Oy, Espoo, Finland) mixture. A drop of 0.5% tropicamid (Santen Oy) was applied on the cornea to dilate the pupils. A drop of oftan obucain (Santen Oy) was used as topical anesthesia. Laser photocoagulation was performed once using a 532-nm diode Oculight® TX laser (Iridex Corp., CA, USA) attached to a slit lamp. A coverslip and Viscotears® gel (Novartis Alcon) were used to applanate the cornea. Three laser lesions were performed in the right eye of each mouse. The anesthesia was immediately reversed by α2-antagonist for medetomidine, atipamezole (5 mg/kg s.c., Antisedan, Orion Pharma, Espoo, Finland).

Test and Reference Compounds

Recombinant human calreticulin protein was produced by UAB Baltymas in yeast Pichia pastoris using recombinant protein production technology based on the secretion of native recombinant protein to the culture medium after expression of human calreticulin precursor including its native signal sequence (Čiplys et al., 2014, 2015). Final protein product having the amino acid sequence of SEQ ID No: 1 was formulated in PBS solution to concentrations of 250 μg/ml, 25 μg/ml, and 2.5 μg/ml (10 mM sodium phosphate, 137 mM sodium chloride and 2.7 mM potassium chloride, pH 7.4) and kept frozen at −80° C. Before the experiment protein solution was thawed and further stored at +4° C. during entire study. The reference compound aflibercept (Eylea®, Bayer Pharma AG) was purchased from the University Pharmacy (Kuopio, Finland) as a ready-to-use solution for intravitreal injections in humans at a concentration of 40 mg/ml and was diluted before use to 20 mg/ml.

Treatment Administration

For groups 1-3 the reference compound (Eylea®), vehicle (PBS) and test compound (calreticulin) were administered unilaterally (right eye) IVT once: immediately after the lasering and in vivo imaging on Day 0. The mice received an injection volume of 2 μl administered using a 5 μl glass microsyringe (Hamilton Bonaduz AG, Bonaduz, Switzerland). For groups 4-7 the vehicle (PBS) and test compounds (calreticulin at the various concentrations) were administered topically, three times per day: 8:00, 13:00 and 18:00. The animals received 5 μl solution into right eye.

In Vivo Imaging

The in vivo imaging using spectral domain optical coherence tomography (SD-OCT) was performed at the baseline to verify the absence of any ocular/retinal pathology, and immediately after the lasering using SD-OCT and fluorescein angiography (FA) to verify the successful damage of Bruch's membrane. The progression of CNV was followed using both SD-OCT and FA on follow-up days 5, 10 and 14.

Fluorescein Angiography

The mice received intraperitoneal injections of 1 ml of 5% fluorescein sodium salt (Sigma-Aldrich Finland Oy, cat. no. F6377). Vascular leakage was examined using Heidelberg Spectralis HRA2 system (Heidelberg Engineering, Germany). Briefly, the mouse was placed into the mouse holder and the imaging system was aligned with the first infrared reflectance image taken with the system. Then, sodium fluorescein was administered. Consecutive FA images were taken every 60 sec from the retinal and choroid focus level for a period of 5 min from sodium fluorescein injection.

Spectral Domain Optical Coherence Tomography

CNV lesions were monitored using Envisu R2200 SD-OCT system (Bioptigen Inc., USA) in mice that had been anaesthetized as described above.

Data Analysis

All image data analysis was performed by blinded evaluators with specialization in ophthalmology and experience in similar studies using ocular mice models. Quantitative data was graphed and analyzed using GraphPad Prism software (v. 8.0, GraphPad Software Inc.).

Data were analyzed using One-Way ANOVA test with Tukey test for multiple comparisons. Differences were considered as statistically significant at the P<0.05 level.

Results:

1) Weight

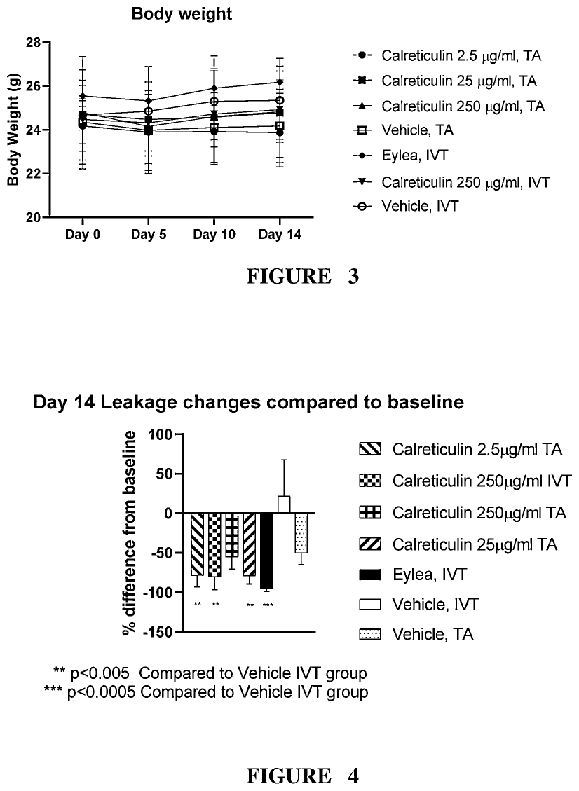

The body weight for the different treatments was similar at the starting of the study and through all study (see Table 1 and FIG. 3).

TABLE 1
The weight of mice from different groups at the baseline
(Day 0) and on every fallow up time point.

| | Weight, g | | | |
|---|---|---|---|---|
| Group | Baseline | Day 5 | Day 10 | Day 14 |
| Group 1 - Vehicle, IVT | 24.675 ± 2.06 | 24.85 ± 2.04 | 25.3 ± 2.08 | 25.35 ± 1.92 |
| Group 2 - Eylea ®, IVT | 25.55 ± 0.48 | 25.325 ± 0.48 | 25.9 ± 0.8 | 26.175 ± 0.51 |
| Group 3- Cal reticulin | 24.475 ± 1.12 | 24.325 ± 1.3 | 24.725 ± 1.18 | 24.925 ± 1.36 |
| 250 μg/ml, IVT | | | | |
| Group 4 - Calreticulin | 24.175 ± 1.15 | 23.9 ± 1.89 | 23.925 ± 1.5 | 23.875 ± 1.58 |
| 2.5 μg/ml, TA | | | | |
| Group 5 - Calreticulin | 24.7 ± 0.72 | 24.475 ± 0.51 | 24.575 ± 0.68 | 24.775 ± 0.71 |
| 25 μg/ml, TA | | | | |
| Group 6 - Calreticulin | 24.78 ± 2.57 | 24.16 ± 2.03 | 24.6 ± 2.19 | 24.82 ± 2.09 |
| 250 μg/ml, TA | | | | |
| Group 7 - Vehicle, TA | 24.35 ± 1.92 | 23.975 ± 1.52 | 24.1 ± 1.59 | 24.175 ± 1.68 |

Data are presented as mean ± SD.

2) CNV

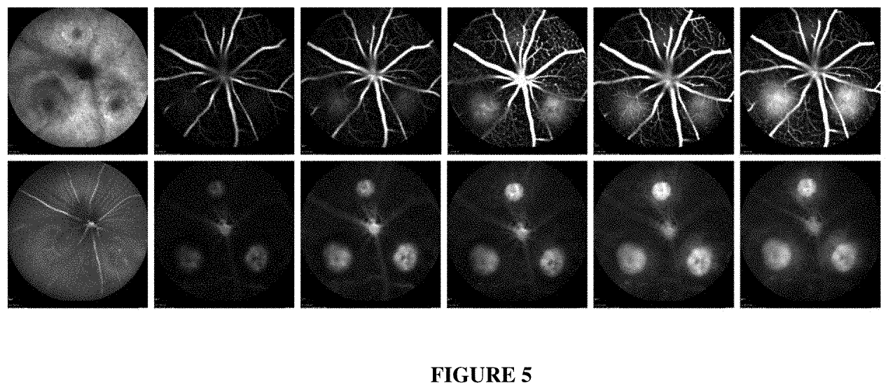

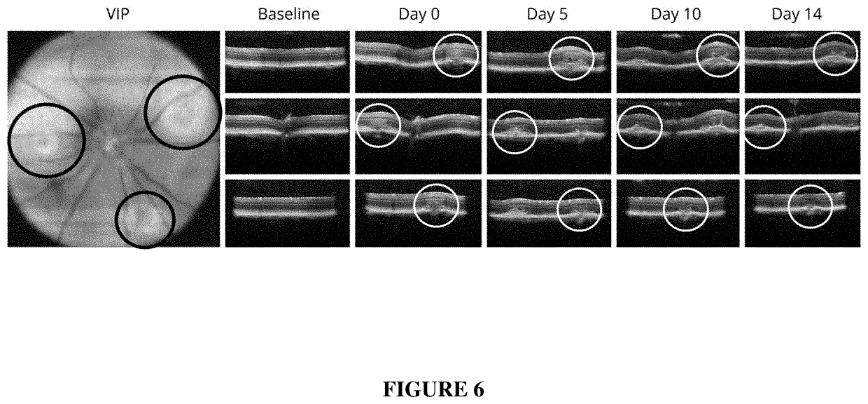

To compare the effect of treatment on CNV, the presence of leaky CNV lesions was graded from FA (FIG. 5) and OCT (FIG. 6) images acquired at the baseline, immediately after CNV induction on day 0, on the follow-up day 5, day 10 and day 14. The percentage of leaky CNVs at a given measurement time point in each treatment group was compared (FIG. 1).

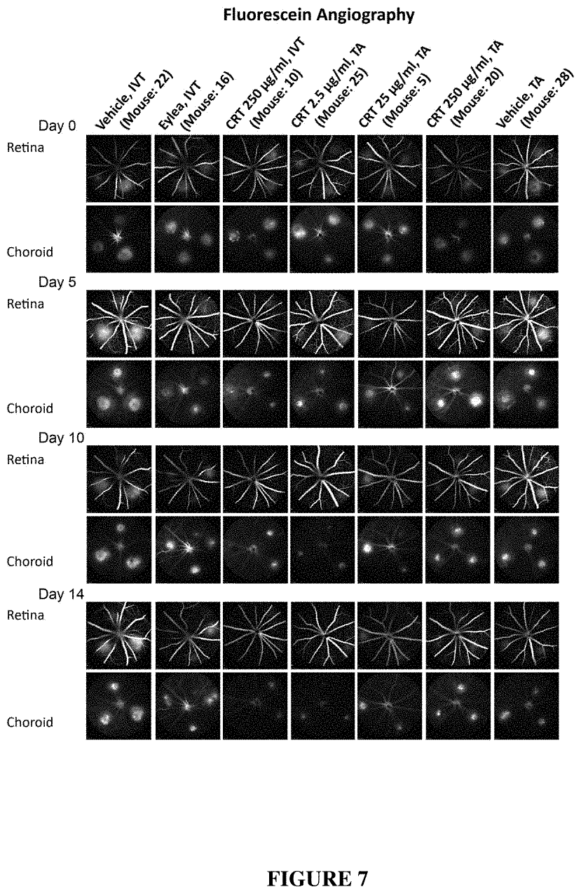

The vascular leakage area was manually outlined from the FA images that were taken from retinal level using Image J software (values for each animal and calculated spots are provided in Table 2). Results were evaluated using GraphPad Prism 8 software. Means and SEM were calculated and significance of the differences between treatment groups assessed by Tukey's multiple comparison test (graph with the results of analysis is provided in FIG. 4). Representative imaging by the FA with examples of animals from each group is shown in FIG. 7.

Similarly as in calculation of the presence of CNV lesions (FIG. 1), IVT treatment with both Eylea® and CRT showed significant effect in comparison to IVT control (vehicle PBS alone), with slightly stronger effects of the Eylea®. TA of two lower concentrations of CRT (2.5 and 25 μg/ml) were similarly effective as IVT injection of CRT (FIG. 4).

TABLE 2
Vascular leakage area in all animals.

| | | Area (mm²) | | | | | | | | | | | |
|---|---|---|---|---|---|---|---|---|---|---|---|---|---|
| | | Day 0 | | | Day 5 | | | Day 10 | | | Day 14 | | |
| Mouse | | Spot | Spot | Spot | Spot | Spot | Spot | Spot | Spot | Spot | Spot | Spot | Spot |
| ID | Treatment | 1 | 2 | 3 | 1 | 2 | 3 | 1 | 2 | 3 | 1 | 2 | 3 |
| 1 | Vehicle, IVT | 0.062 | 0.046 | 0.064 | 0.034 | 0.020 | 0.076 | 0.031 | 0.014 | 0.047 | 0.034 | 0.023 | 0.025 |
| 2 | Eylea ®, IVT | 0.042 | 0.042 | 0.048 | 0.000 | 0.000 | 0.000 | 0.000 | 0.000 | 0.057 | 0.000 | 0.000 | 0.000 |
| 3 | Calreticulin 250 | 0.009 | 0.041 | 0.071 | 0.000 | 0.024 | 0.058 | 0.000 | 0.028 | 0.064 | 0.000 | 0.000 | 0.000 |
| | μg/ml, IVT | | | | | | | | | | | | |
| 4 | Calreticulin 2.5 | 0.048 | 0.021 | 0.024 | 0.030 | 0.054 | 0.032 | 0.000 | 0.072 | 0.056 | 0.000 | 0.053 | 0.047 |
| | μg/ml, TA | | | | | | | | | | | | |
| 5 | Calreticulin 25 | 0.025 | 0.050 | 0.029 | 0.029 | 0.021 | 0.026 | 0.000 | 0.000 | 0.033 | 0.000 | 0.019 | 0.015 |
| | μg/ml, TA | | | | | | | | | | | | |
| 6 | Calreticulin 250 | 0.014 | 0.034 | 0.007 | 0.067 | 0.086 | 0.084 | 0.026 | 0.092 | 0.055 | 0.030 | 0.061 | 0.039 |
| | μg/ml, TA | | | | | | | | | | | | |
| 7 | Vehicle, TA | 0.042 | 0.048 | 0.016 | 0.024 | 0.027 | 0.000 | 0.000 | 0.000 | 0.000 | 0.000 | 0.000 | 0.000 |
| 8 | Vehicle, IVT | 0.011 | 0.057 | 0.006 | 0.042 | 0.058 | 0.000 | 0.021 | 0.095 | 0.000 | 0.000 | 0.000 | 0.000 |
| 9 | Eylea ®, IVT | 0.042 | 0.047 | 0.036 | 0.000 | 0.000 | 0.000 | 0.117 | 0.019 | 0.000 | 0.000 | 0.000 | 0.000 |
| 10 | Calreticulin 250 | 0.043 | 0.055 | 0.031 | 0.000 | 0.000 | 0.000 | 0.000 | 0.000 | 0.000 | 0.000 | 0.000 | 0.000 |
| | μg/ml, IVT | | | | | | | | | | | | |
| 11 | Calreticulin 2.5 | 0.053 | 0.062 | 0.025 | 0.025 | 0.072 | 0.016 | 0.000 | 0.000 | 0.000 | 0.000 | 0.000 | 0.000 |
| | μg/ml, TA | | | | | | | | | | | | |
| 12 | Calreticulin 25 | 0.031 | 0.032 | 0.053 | 0.020 | 0.040 | 0.111 | 0.000 | 0.000 | 0.000 | 0.000 | 0.000 | 0.000 |
| | μg/ml, TA | | | | | | | | | | | | |
| 13 | Calreticulin 250 | 0.059 | 0.047 | 0.036 | 0.103 | 0.148 | 0.025 | 0.044 | 0.081 | 0.028 | 0.051 | 0.000 | 0.000 |
| | μg/ml, TA | | | | | | | | | | | | |
| 14 | Vehicle, TA | 0.023 | 0.030 | 0.051 | 0.009 | 0.007 | 0.040 | 0.011 | 0.029 | 0.009 | 0.033 | 0.044 | 0.035 |
| 15 | Vehicle, IVT | 0.008 | 0.018 | 0.014 | 0.015 | 0.083 | 0.042 | 0.055 | 0.081 | 0.012 | 0.057 | 0.115 | 0.011 |
| 16 | Eylea ®, IVT | 0.039 | 0.073 | 0.046 | 0.000 | 0.078 | 0.000 | 0.027 | 0.006 | 0.040 | 0.022 | 0.006 | 0.000 |
| 17 | Calreticulin 250 | 0.038 | 0.021 | BS* | 0.048 | 0.108 | — | 0.042 | 0.062 | — | 0.015 | 0.068 | — |
| | μg/ml, IVT | | | | | | | | | | | | |
| 18 | Calreticulin 2.5 | 0.046 | 0.021 | 0.038 | 0.031 | 0.075 | 0.030 | 0.017 | 0.077 | 0.071 | 0.000 | 0.000 | 0.000 |
| | μg/ml, TA | | | | | | | | | | | | |
| 19 | Calreticulin 25 | 0.018 | 0.026 | 0.038 | 0.034 | 0.013 | 0.000 | 0.000 | 0.000 | 0.000 | 0.000 | 0.000 | 0.000 |
| | μg/ml, TA | | | | | | | | | | | | |
| 20 | Calreticulin 250 | 0.034 | 0.023 | 0.078 | 0.024 | 0.024 | 0.030 | 0.009 | 0.017 | 0.005 | 0.021 | 0.038 | 0.021 |
| | μg/ml, TA | | | | | | | | | | | | |
| 21 | Vehicle, TA | 0.033 | 0.056 | 0.046 | 0.025 | 0.014 | 0.030 | 0.018 | 0.000 | 0.018 | 0.036 | 0.000 | 0.049 |
| 22 | Vehicle, IVT | 0.034 | 0.076 | 0.007 | 0.037 | 0.129 | 0.124 | 0.040 | 0.100 | 0.075 | 0.035 | 0.131 | 0.059 |
| 23 | Eylea ®, IVT | 0.034 | 0.054 | 0.023 | 0.035 | 0.077 | 0.047 | 0.000 | 0.042 | 0.027 | 0.000 | 0.000 | 0.000 |
| 24 | Calreticulin 250 | 0.046 | 0.043 | BS* | 0.052 | 0.023 | — | 0.000 | 0.000 | — | 0.000 | 0.000 | — |
| | μg/ml, IVT | | | | | | | | | | | | |
| 25 | Calreticulin 2.5 | 0.048 | 0.065 | 0.011 | 0.000 | 0.074 | 0.041 | 0.000 | 0.000 | 0.000 | 0.000 | 0.000 | 0.000 |
| | μg/ml, TA | | | | | | | | | | | | |
| 27 | Calreticulin 250 | 0.027 | 0.052 | 0.052 | 0.000 | 0.000 | 0.000 | 0.000 | 0.000 | 0.000 | 0.000 | 0.000 | 0.000 |
| | μg/ml, TA | | | | | | | | | | | | |
| 28 | Vehicle, TA | 0.043 | 0.094 | 0.036 | 0.033 | 0.066 | 0.038 | 0.025 | 0.054 | 0.044 | 0.000 | 0.024 | 0.036 |
| 29 | Calreticulin 25 | 0.044 | 0.040 | 0.035 | 0.000 | 0.023 | 0.000 | 0.044 | 0.027 | 0.000 | 0.038 | 0.016 | 0.000 |
| | μg/ml, TA | | | | | | | | | | | | |
| 30 | Calreticulin 250 | 0.041 | 0.037 | 0.043 | 0.017 | 0.020 | 0.050 | 0.000 | 0.000 | 0.000 | 0.000 | 0.000 | 0.000 |
| | μg/ml, TA | | | | | | | | | | | | |

(*BS = bad shot)

### Example 2

This example describes stability studies performed with recombinant CRT protein preparations derived from yeast P. pastoris in order to define optimal pH and buffer composition for formulation and long-term storage of therapeutic preparations containing a full-length CRT protein in solution for application onto the eyes, and to test stability of the preparations after freezing and thawing.

For the use of protein as a drug substance, a long-term stability of its preparations is of critical importance. Topical application of the protein in the form of eye drops requires stability in solution at working concentrations. Previous studies using N-terminal CRT fragment vasostatin (1-180 amino acids of CRT protein) for topical applications on animal eyes included its stability analysis in the solution. According to the reports, vasostatin was stable for seven days when stored at 4° C. at working concentrations either in methylcellulose solution or in PBS (Wu et al, 2005; Sheu et al., 2009). It is evident from published results (FIG. 3 in Wu et al, 2005) that longer storage resulted in a loss of vasostatin integrity (analysed by SDS-PAGE method) and, accordingly, in a continuous decrease of anti-angiogenic activity after the second week storage. Obviously, such stability time period is too short to develop convenient therapy, as even treatment course of CNV in the mouse model took three weeks (Sheu et al., 2009) let alone sufficient time periods needed for storage of therapeutic preparations before their use. Therefore, stability of vasostatin preparations would require significant improvement by adding specific stabilizers or changing buffer composition, pH, etc. It is not clear if such manipulations would be compatible with intended use for the eye treatment. Tai et al., 2013 tried to solve this and other problems related to application of vasostatin by reducing the size of expressed vasostatin fragment, as described in the Background section.

### Example 2-1

First, we analysed stability of the recombinant CRT protein in solution at different pH. CRT protein having SEQ ID No: 1 purified from P. pastoris was transferred into solutions with 0.1 M final concentrations of appropriate buffers at different pH in the range from 4 to 9 and incubated at different temperatures (22° C., 37° C. and 45° C.) at final protein concentration of 0.2 mg/ml. CRT has aggregated at pH 4.0 (succinate buffer), therefore this pH was discarded from further analysis as inappropriate. Protein samples from pH 5 to 9 were taken after different time points at different temperatures and were analysed by densitometrical scanning of SDS-PAGE gels (gels were scanned by densitometric scanner BIO-5000 PLUS and images were analysed using ImageQuant TL 8.1 software). The percentage of intact calreticulin was calculated by comparison to CRT protein sample taken before incubation (at 0 hour time point) and run as a control in each SDS-PAGE gel. This initial study was carried out for 3 months.

The results showed that at a room temperature (+22° C.) CRT is generally stable in the pH range from 7 to 8, where the amount of intact CRT form after 3 months was determined to be ˜90% or higher (SDS-PAGE gel with CRT samples is shown in FIG. 8). Densitometric evaluation of the bands in scanned gels is not a very accurate analysis method and shows up to 10% measuring errors, therefore we consider that the protein remained stable if the amount of intact CRT form was calculated to be 90% or more in comparison to initial control sample.

Incubation at pH 5-6 resulted in significant CRT degradation, whereas after incubation at pH 9 (FIG. 8, lane 6) the amount of remaining intact CRT form was calculated to be ˜80%. Differences in protein stability between samples in the pH interval 7-9 after incubation at room temperature were not very clear, therefore the analysis after incubation at higher temperature was more informative.

It should be noted that CRT is not stable at the higher than physiological temperatures. Previously it was demonstrated that native CRT isolated from human placenta oligomerizes at temperatures above 40° C. (Jørgensen et al., 2003). Similarly, recombinant CRT has also been reported to oligomerize/polymerize at 37-45° C. (Mancino et al., 2002). In addition to oligomerization, we have noticed that both native human placental CRT and recombinant CRT from yeast are prone to partial degradation after longer incubation at the elevated temperatures. Actually, oligomerization coincides with increased partial degradation of CRT, and this may also be illustrated by FIG. 8. In our hands, it was enough to keep protein sample for 3 min at 85° C. to get completely oligomerized CRT (examined by native PAGE). After such heat shock oligomerized CRT has partially degraded even during incubation at room temperature (FIG. 8, lane 9), whereas degradation of monomeric CRT in the corresponding sample without pre-heating at 85° C. was negligible (FIG. 8, lane 8).

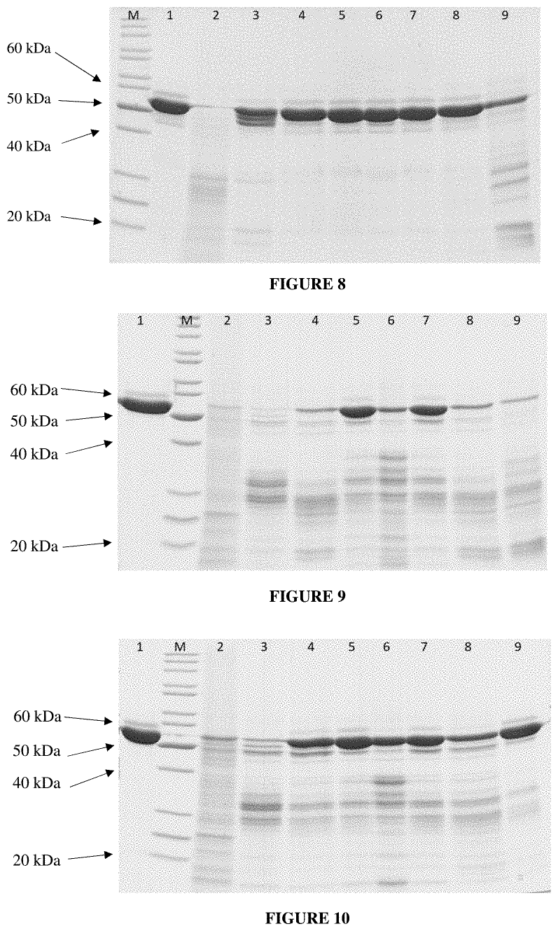

Incubation of CRT at higher than physiological 45° C. temperature resulted in a rapid partial degradation of the protein in all pH values tested, with no intact CRT form detected already after few or several days. Therefore, the most informative indication of protein stability was its incubation at 37° C. Initial analysis at this temperature showed that CRT was the most stable in Tris buffer, at pH 7.5-8.0 (FIG. 9, lanes 5 and 7 in comparison to the lane 6 with CRT in Tris buffer, pH 9.0). It is noticeable that the protein was much less stable in a very similar pH 7.4 in a phosphate buffer. Further analysis showed that the concentration of buffering phosphate has specific effect on CRT stability. We did experiments in phosphate buffer without NaCl and results showed that CRT is stabilized in a range of concentrations from approximately 7 mM to ˜17 mM. Increasing phosphate concentration to 20 mM or more resulted in appearance of noticeable degradation of CRT. Therefore, we consider that using phosphate buffer for formulation of stable CRT solution, the concentration of buffering phosphate salt should be in a range of 5-20 mM. Similar results were observed with either the pH 7.2 or 7.4. Thus, recommended pH of the phosphate buffer for stable CRT solution would be in a range of 7.0-7.6.

Addition of saline (NaCl) at physiological concentrations to the phosphate buffer had no significant effect on CRT stability.

Improved stability of the CRT at 10 mM phosphate concentration is shown in FIG. 10 with an example of SDS-PAGE analysis after 9-10 days incubation of protein samples at 37° C. (lane 9 with CRT incubated in 10 mM phosphate vs. similar CRT sample with 100 mM phosphate in lane 8).

In the case of Tris buffer we did not notice difference in CRT stability either using 20 mM or 100 mM Tris concentrations. Therefore, possible concentrations of Tris in stable working CRT protein solution may be from 10 mM to 130 mM with buffer pH interval from 7.0 to 8.5.

As the physiological pH of tear fluid is 7.4 (Agrahari et al., 2016), it is convenient to formulate CRT solution for the eye drops as close to this pH as possible by using either phosphate or Tris buffers. Both phosphate and Tris are appropriate buffers for drug development according to US and European Pharmacopoeia requirements.

### Example 2-2

We have conducted initial freezing/thawing experiments with recombinant CRT protein preparations derived from yeast P. Pastoris as per Example 1.

At the time of production, we have compared stability of final purified protein preparations in Tris buffer solution without freezing vs. frozen at −80° C. for storage and thawed for further use. No difference in protein integrity was noticed between non-frozen and frozen/thawed CRT samples in solution during their incubation for 3 months at 5° C., 22° C. or 37° C. followed by examination using electrophoresis methods (SDS-PAGE and native PAGE).

Frozen CRT samples were further tested by several analytical methods developed according to requirements applied for analysis of the drug substance. Purity and homogeneity of CRT protein preparations were found to be in a level sufficient even for the development of injectable pharmaceutical preparations.

A second freeze/thaw cycle by additional freezing at either −80° C. or −20° C. was performed and the analytical testing procedures repeated. The same results were obtained after the second freeze/thaw cycle of CRT preparation in Tris buffer using the main analytical methods RP-HPLC, SE-HPLC, SDS-PAGE and native PAGE. These results suggest that CRT protein does not degrade or oligomerize/polymerize after additional cycle of freezing/thawing.

### Example 2-3

For the longer stability study we have chosen simple PBS (phosphate buffered saline) buffer, because its pH (7.4) and physiological saline concentration seems to be optimal for topical ocular use. The same CRT protein formulations in PBS used for in vivo study in the mouse CNV model (described in Example 1) were also tested for their long-term stability at different temperatures.

As noted there, CRT protein having SEQ ID NO: 1 (that had been previously frozen after production/purification and then thawed) was formulated in PBS (10 mM sodium phosphate, 137 mM sodium chloride and 2.7 mM potassium chloride, pH 7.4) at three working concentrations of 250 μg/ml, 25 μg/ml, and 2.5 μg/ml. Prepared CRT solutions were filter-sterilized, aliquoted into multiple tubes and frozen again at −80° C. After storage all tubes were thawed at approximately the same date and part of them were used in the in vivo experiments in the mouse CNV model (Example 1), whereas other tubes were divided into five groups and further incubated for the stability study at different temperatures: −80° C., −20° C. (storage in frozen state; the third freezing cycle in total), 5° C., 22° C. and 37° C. (storage in solution). Samples were taken for analysis of protein integrity by SDS-PAGE at different time points during a period of 9 months.

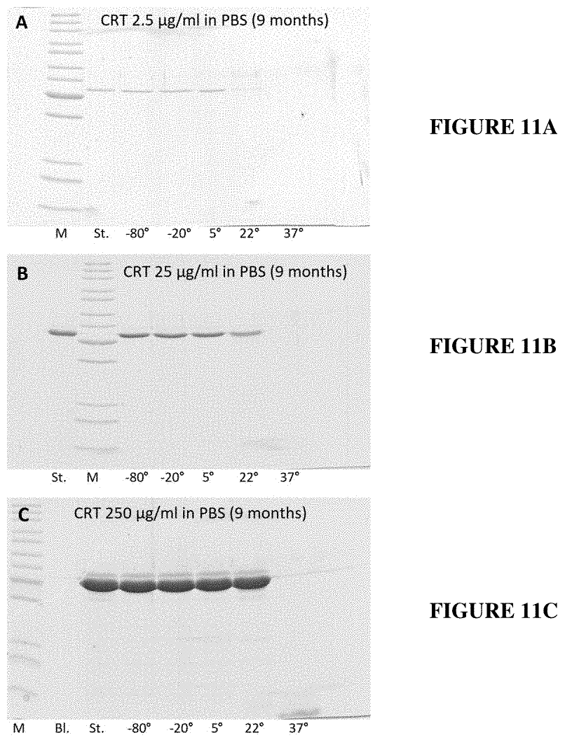

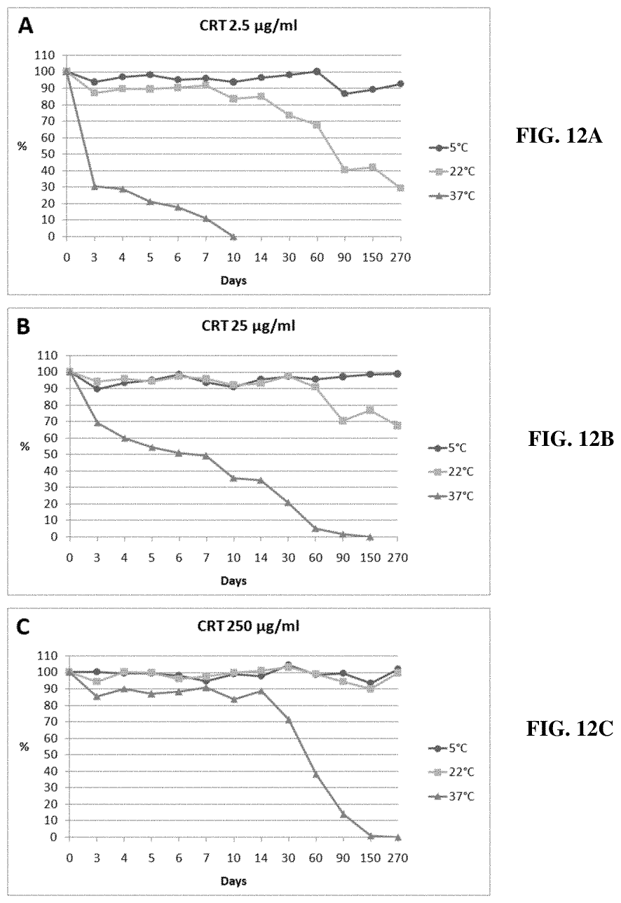

Examples of SDS-PAGE gels with CRT from preparations of different protein concentrations after 9 months incubation is shown in FIGS. 11A to 11C. Calculated percentages of intact CRT form at each time point of entire long-term study is provided in FIGS. 12A to 12C. The results show that CRT is extremely stable in PBS solution at the higher protein concentration of 250 g/ml when incubated at either 5° C. or 22° C. temperatures (FIG. 11C and FIG. 12C). Diluted CRT solutions appeared to be less stable, nevertheless at both 25 μg/ml and 2.5 μg/ml concentrations protein still remained intact after 9 months incubation at the lower temperature of 5° C. (FIGS. 11A and 11B and FIGS. 12A and 12B). It suggests that CRT formulated for topical application in a form of eye drops using simple PBS can be stored at 4-5° C. in solution without any additives and remain stable and functional at diluted working concentrations for at least 9 months. It seems sufficient stability for the development of a new topically applied medicine containing recombinant CRT.

The results or Examples 2-2 and 2-3 also suggest that long-term storage periods of diluted CRT solutions can be greatly extended by simply freezing final protein preparations at −20° C. The study in Example 1 was done using CRT protein after freezing in PBS at working concentrations (in total, it was the second freezing-thawing of the used CRT protein batch). The third freezing of CRT in stability study described here in Example 2-3 preserved the intact stable protein after the storage at either −80° C. or −20° C. (FIGS. 11A to 11C).

Taking all together, the results show that recombinant CRT protein preparations can be repeatedly frozen for a long-term storage for years without the loss of protein integrity and activity.

The results described in Example 2 demonstrates that present invention not only shows unexpected therapeutic effect of the CRT protein after intravitreal and topical application, but also provides solution for required long-term stability of active protein substance, particularly in formulations of eye drops.

The examples described herein are to be understood as illustrative examples of embodiments of the invention. Further embodiments and examples are envisaged. Any feature described in relation to any one example or embodiment may be used alone or in combination with other features. In addition, any feature described in relation to any one example or embodiment may also be used in combination with one or more features of any other of the examples or embodiments, or any combination of any other of the examples or embodiments. Furthermore, equivalents and modifications not described herein may also be employed within the scope of the invention, which is defined in the claims.

### REFERENCES

Agrahari V, Mandal A, Agrahari V, Trinh H M, Joseph M, Ray A, Hadji H, Mitra R, Pal D, Mitra A K: A comprehensive insight on ocular pharmacokinetics. Drug Deliv Transl Res. 2016 December; 6(6):735-754.
Barar J, Javadzadeh A R, Omidi Y: Ocular novel drug delivery: impacts of membranes and barriers. Expert Opin Drug Deliv. 2008 May; 5(5):567-81.
Bee Y S, Ma Y L, Chen J, Tsai P J, Sheu S J, Lin H C, Huang H, Liu G S, Tai M H: Inhibition of Experimental Choroidal Neovascularization by a Novel Peptide Derived from Calreticulin Anti-Angiogenic Domain. Int J Mol Sci. 2018 Sep. 30; 19 (10). pii: E2993.
Bee Y S, Sheu S J, Ma Y L, Lin H C, Weng W T, Kuo H M, Hsu H C, Tang C H, Liou J C, Tai M H: Topical application of recombinant calreticulin peptide, vasostatin 48, alleviates laser-induced choroidal neovascularization in rats. Mol Vis. 2010 Apr. 28; 16:756-67.
Chen C N, Chang C C, Su T E, Hsu W M, Jeng Y M, Ho M C, Hsieh F J, Lee P H, Kuo M L, Lee H, Chang K J: Identification of calreticulin as a prognosis marker and angiogenic regulator in human gastric cancer. Ann Surg Oncol. 2009 February; 16(2):524-33.
Čiplys E, Slibinskas R, Sasnauskas K, Michalak M, Gold L I: Generation of native recombinant secreted human endoplasmic reticulum chaperones by using their native signal sequences in yeast expression systems. PCT patent application W O 2014/011723 A1. Pub. Date: 2014 Jan. 16.
Čiplys E, Žitkus E, Gold L I, Daubriac J, Pavlides S C, Hojrup P, Houen G, Wang W A, Michalak M, Slibinskas R: High-level secretion of native recombinant human calreticulin in yeast. Microb Cell Fact. 2015 Oct. 15; 14:165.
Eggleton P, Bremer E, Dudek E, Michalak M: Calreticulin, a therapeutic target? Expert Opin Ther Targets. 2016 September; 20(9):1137-47.
Friis T, Kjaer Sørensen B, Engel A M, Rygaard J, Houen G: A quantitative ELISA-based co-culture angiogenesis and cell proliferation assay. APMIS. 2003 June; 111(6):658-68.
Gaudana R, Ananthula H K, Parenky A, Mitra A K: Ocular drug delivery. AAPS J. 2010 September; 12(3):348-60.
Gillies M C, Campain A, Barthelmes D, Simpson J M, Arnold J J, Guymer R H, McAllister I L, Essex R W, Morlet N, Hunyor A P; Fight Retinal Blindness Study Group: Long-Term Outcomes of Treatment of Neovascular Age-Related Macular Degeneration: Data from an Observational Study. Ophthalmology. 2015 September; 122(9):1837-45.
Jørgensen C S, Ryder L R, Steinø A, Højrup P, Hansen J, Beyer N H, Heegaard N H, Houen G: Dimerization and oligomerization of the chaperone calreticulin. Eur J Biochem. 2003 October; 270(20):4140-8.
Mancino L, Rizvi S M, Lapinski P E, Raghavan M: Calreticulin recognizes misfolded HLA-A2 heavy chains. Proc Natl Acad Sci USA. 2002 Apr. 30; 99(9):5931-6.
Pike S E, Yao L, Jones K D, Cherney B, Appella E, Sakaguchi K, Nakhasi H, Teruya-Feldstein J, Wirth P, Gupta G, Tosato G: Vasostatin, a calreticulin fragment, inhibits angiogenesis and suppresses tumor growth. J Exp Med. 1998 Dec. 21; 188(12):2349-56.
Pike S E, Yao L, Setsuda J, Jones K D, Cherney B, Appella E, Sakaguchi K, Nakhasi H, Atreya C D, Teruya-Feldstein J, Wirth P, Gupta G, Tosato G: Calreticulin and calreticulin fragments are endothelial cell inhibitors that suppress tumor growth. Blood. 1999 Oct. 1; 94(7):2461-8.
Sheng W, Chen C, Dong M, Zhou J, Liu Q, Dong Q, Li F: Overexpression of calreticulin contributes to the development and progression of pancreatic cancer. J Cell Physiol. 2014 July; 229(7):887-97.
Sheu S J, Bee Y S, Ma Y L, Liu G S, Lin H C, Yeh T L, Liou J C, Tai M H: Inhibition of choroidal neovascularization by topical application of angiogenesis inhibitor vasostatin. Mol Vis. 2009 Sep. 18; 15:1897-905.
Tai M H, Bee Y S, Chen S C: Medication and therapy treating of choroidal neovascularization. U.S. patent application No. U.S. 2013/0296242 A1. Pub. Date: Nov. 7, 2013.
Tosato G, Pike S E, Yao L: Use of calreticulin and calreticulin fragments to inhibit endothelial cell growth and angiogenesis, and suppress tumor growth. U.S. 2005/0208018 A1. Pub. Date: Sep. 22, 2005.
Wecker T, Ehlken C, Bühler A, Lange C, Agostini H, Böhringer D, Stahl A: Five-year visual acuity outcomes and injection patterns in patients with pro-re-nata treatments for AMD, DME, RVO and myopic CNV. Br J Ophthalmol. 2017 March; 101(3):353-359.
Wu P C, Yang L C, Kuo H K, Huang C C, Tsai C L, Lin P R, Wu P C, Shin S J, Tai M H: Inhibition of corneal angiogenesis by local application of vasostatin. Mol Vis. 2005 Jan. 13; 11:28-35.

Sequences:

SEQ ID No: 1 as used herein has the following sequence (amino acids 18-417 of human calreticulin precursor shown in UniProtKB Database Id. No. P27797):

EPAVYFKEQFLDGDGWTSRWIESKHKSDFGKFVLSSGKFYGDEEKDKGLQ
TSQDARFYALSASFEPFSNKGQTLVVQFTVKHEQNIDCGGGYVKLFPNSL
DQTDMHGDSEYNIMFGPDICGPGTKKVHVIFNYKGKNVLINKDIRCKDDE
FTHLYTLIVRPDNTYEVKIDNSQVESGSLEDDWDFLPPKKIKDPDASKPE
DWDERAKIDDPTDSKPEDWDKPEHIPDPDAKKPEDWDEEMDGEWEPPVIQ
NPEYKGEWKPRQIDNPDYKGTWIHPEIDNPEYSPDPSIYAYDNFGVLGLD
LWQVKSGTIFDNFLITNDEAYAEEFGNETWGVTKAAEKQMKDKQDEEQRL
KEEEEDKKRKEEEEAEDKEDDEDKDEDEEDEEDKEEDEEEDVPGQAKDEL

1. A method for treating or preventing angiogenic eye disease in a subject, comprising administering a composition comprising calreticulin to a subject in need thereof.

2. The method according to claim 1, wherein the angiogenic eye disease is selected from the group consisting of choroidal neovascularization, corneal neovascularization, retinal neovascularization, and combinations thereof.

3. The method according to claim 1, wherein the angiogenic eye disease is selected from the group consisting of proliferative diabetic retinopathy, macular degeneration, and neovascular glaucoma.

4. The method according to claim 3, wherein the angiogenic eye disease is age-related macular degeneration.

5. The method according to claim 1, wherein the calreticulin prevents or reduces neovascularization.

6. The method according to claim 1, wherein the subject is a human and the calreticulin is human calreticulin.

7. The method according to claim 1, wherein the calreticulin has the amino acid sequence of SEQ ID NO: 1 or an amino acid sequence having at least 85% sequence identity to the amino acid sequence of SEQ ID NO: 1.

8. The method according to claim 1, wherein the calreticulin is a mature full-length protein.

9. The method according to claim 1, wherein the calreticulin is obtained from eukaryotic cells.

10. The method according to claim 9, wherein the calreticulin is obtained by recombinant expression in yeast.

11. The method according to claim 1, wherein at least 75% of the calreticulin in the composition is in monomeric form.

12. The method according to claim 1, wherein said administering is via intravitreal injection into the eye.

13. The method according to claim 12, wherein the concentration of calreticulin in the composition is 25 μg/ml to 50 mg/ml.

14. The method according to claim 1, wherein said administering is to the eye of said subject via topical application.

15. The method according to claim 14, wherein the concentration of calreticulin in the composition is 25 ng/ml to 250 μg/ml.

16. The method according to claim 12, wherein the subject is treated via topical application to the eye with said composition prior to said intravitreal injection.

17. The method according to claim 14, wherein the subject is treated by intravitreal injection prior to said topical application.

18. The method according to claim 1, wherein said administering is combined, separate, or sequential to administration with an anti-angiogenic agent.

19. The method according to claim 18, wherein the anti-angiogenic agent is an inhibitor of vascular endothelial growth factor (VEGF).

20. A pharmaceutical composition comprising calreticulin and one or more buffering agents, wherein the pharmaceutical composition is formulated for ocular administration.

21. The pharmaceutical composition according to claim 20, where-in the pharmaceutical composition is formulated for topical administration to the eye as eye drops or for intravitreal injection into the eye.

22. The pharmaceutical composition according to claim 20, wherein:
the calreticulin has the amino acid sequence of SEQ ID NO: 1 or an amino acid sequence having at least 85% sequence identity to the amino acid sequence of SEQ ID NO: 1, and/or
the concentration of calreticulin in the composition is 25 μg/ml to 50 mg/ml.

23. A pre-filled intravitreal syringe comprising the pharmaceutical composition according to claim 20.

24. An eye drop bottle comprising the pharmaceutical composition according to claim 20 and a nozzle for dispensing a metered dose of the pharmaceutical composition to the eye.

25. A method of producing a pharmaceutical composition formulated for ocular administration according to claim 20 comprising the steps of:
(a) transforming a yeast cell with a nucleotide sequence comprising a coding sequence encoding a polypeptide comprising a native calreticulin signal sequence and a calreticulin;
(b) culturing the yeast cell under conditions such that the calreticulin is expressed in secreted form;
(c) extracting the calreticulin from the culture medium;
(d) using the calreticulin to make the pharmaceutical composition formulated for ocular administration.
